(19) United States
(12) Patent Application Publication
(10) Pub. No.: US 2009/0131688 A1
(43) Pub. Date: May 21, 2009

(54) METHOD FOR THE SYNTHESIS OF 4-BENZOFURAN-CARBOXYLIC ACID

(75) Inventors: Alain Burgos, Les Ponts De Ce (FR); Martine Maruani, Montreuil Juigne (FR); Florence Perrin, Avrille (FR); Stephane Frein, St. Aubin De Luigne (FR)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: ZACH SYSTEM, Avrille (FR)

(21) Appl. No.: 12/036,350

(22) Filed: Feb. 25, 2008

(30) Foreign Application Priority Data
Nov. 21, 2007 (FR) 0759188

(51) Int. Cl. C07D 311/76; C07D 307/77; C07C 49/76

(52) U.S. Cl. 549/283; 549/470; 568/327; 549/466

(57) ABSTRACT

The invention concerns a novel method for synthesis of 4-benzofuran-carboxylic acid or alkyl ester thereof.

This method is characterized in that a reaction for aromatization of a compound of formula (II) is performed for the synthesis of the compound of formula (I), according to the scheme A2 below:

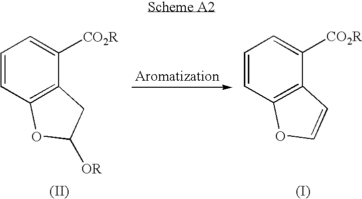

wherein R independently represents hydrogen or a linear or branched C₁-₁₅ alkyl group.

With the invention, it is possible to industrially synthesize 4-benzofuran-carboxylic acid or alkyl ester thereof with good yield and very good purity.

### FIELD OF THE INVENTION

The invention relates to a method for the synthesis of the alkyl 4-benzofuran-carboxylate compound of formula (I) for preparing 4-benzofuran-carboxylic acid.

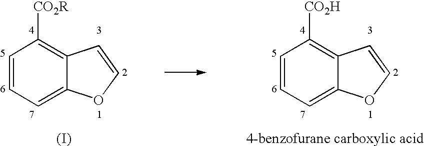

According to the invention, the method for synthesizing the compound of formula (I) is performed in 5 steps, the first 4 of which are carried out without isolating the reaction medium from 4-hydroxyindanone. This method successively applies a ketone silylation reaction, an ozonolysis reaction on the silylated enol ether compound, an oxidation reaction, an esterification reaction, and finally an aromatization reaction for obtaining the compound of formula (I).

The compound of formula (I) is used in a saponification reaction for obtaining 4-benzofuran-carboxylic acid.

### STATE OF THE ART

The article published in the Journal of Medicinal Chemistry, 1995, 38(18), p 3095 of the authors, Eissenstat Michael et al. is known from the prior art, which describes a 4-step method for synthesizing the compound of formula (I), from methyl 4-hydroxybenzoate, by successively applying an etherification reaction, a Claisen rearrangement of an allyl ether hydroxybenzoate, an ozonolysis reaction, and a dehydration reaction.

The compound of formula (I) is then saponified as described below.

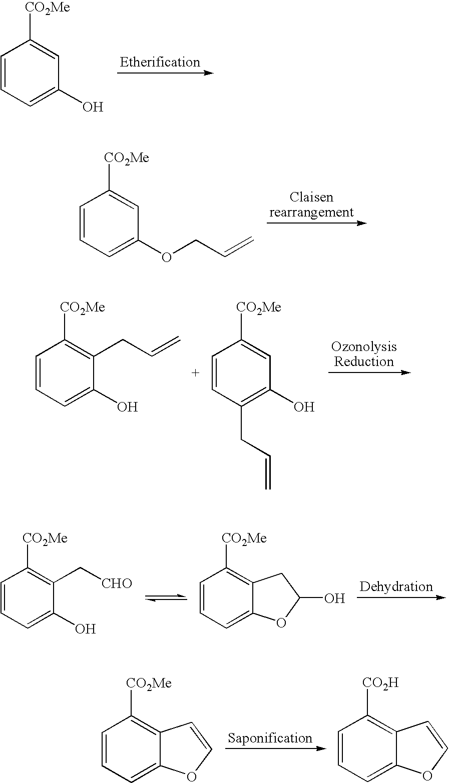

This method has the drawbacks of applying a Claisen rearrangement reaction requiring high temperatures (>200° C.), obtaining a byproduct corresponding to a isomer of position of the para allyl group of the ester function, the latter according to the method leads to the 6-position isomer corresponding to 6-benzofuran-carboxylic acid, which is very difficult to remove from the final product, 4-benzofuran-carboxylic acid.

Finally, the compound of formula (I) is obtained with an overall yield of the order 21%.

Among the other methods known from the literature, the following may be mentioned:

European Patent EP 1739103 (30.06.2005) of the Borealis Technology group claims an ene-silylation method on 4-hydroxy-indanone in the presence of the reagent (R′)₃SiY with R′ representing an either identical or different C1-6 hydrocarbon group, and Y represents a leaving group known to the skilled practitioner, such as a halogen atom, a tosyl, alkoxy or mesyl group, according to the method below.

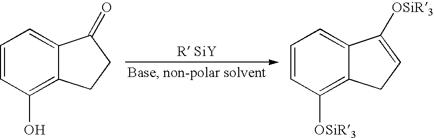

These silylated compounds are used for the synthesis of metal complexes, the latter being catalysts for polymerizing olefins.

European patent EP 240859 (26.03.1987) of the Abbott group which mentions the formation of a silylated enol ether from an indanone, an ozonolysis reaction of the silylated enol ether compound according to the method below.

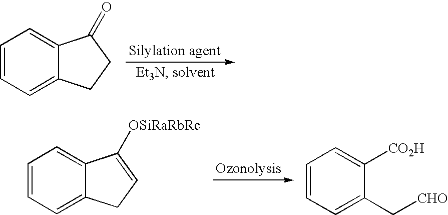

On the indanone compound, the yield for obtaining the silylated enol ether is not known, and the product from the ozonolysis reaction is obtained with a yield of 62%.

This patent in the description of the invention suggests the application of this method on the 4-hydroxy-indanone compound according to the method below,

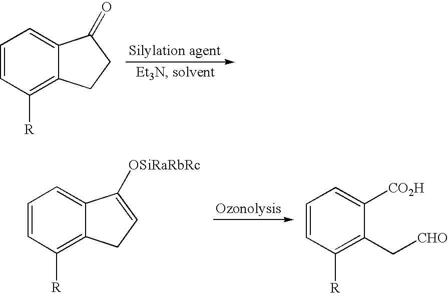

with R representing a hydroxyl (OH).

The feasibility of this method when R represents a hydroxyl is not demonstrated exemplarily.

### OBJECTS OF THE INVENTION

The main object of the present invention is to solve the novel technical problem consisting in providing 4-benzofuran-carboxylic acid as a synthesis intermediate for the pharmaceutical industry according to an industrially profitable method.

The main object of the present invention further is to solve this novel technical problem by applying a novel synthesis method, advantageously without isolating intermediates, thereby improving the productivity for making the product of formula (I), a key intermediate for obtaining 4-benzofuran-carboxylic acid.

The main object of the present invention further is to solve this novel technical problem according to a solution with which 4-benzofuran carboxylic acid may be obtained with an overall chemical yield significantly larger than in the state of the art, i.e., 62%, and a grade of chemical purity with very high titer, larger than 99%, essentially without any contamination by residual synthesis substances, incompatible with pharmaceutical use, in particular 4-benzofuran-carboxylic acid.

### DETAILED DESCRIPTION OF THE INVENTION

For the first time, the present invention has solved the whole of the technical problems mentioned above in a simple way and transposable to an industrial scale, in particular in the pharmaceutical industry.

Thus, according to a first aspect, the present invention provides a method for the synthesis of 4-benzofuran-carboxylic acid or alkyl ester thereof characterized in that an aromatization reaction of a compound of formula (II) is performed for the synthesis of the compound of formula (I), according to the scheme A2 below:

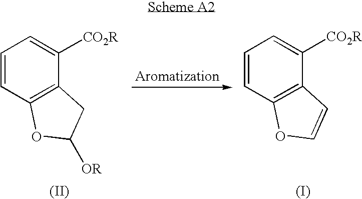

wherein R independently represents a hydrogen or a linear or branched C₁₋₁₅ alkyl group.

According to a particular embodiment, this method is characterized in that the compound of formula (II) is prepared by a reaction for esterification of a compound of formula (III) or of a mixture of compounds with formulae (III), (IV) and (II), stemming from a prior reaction, by adding an acid to the reaction medium, according to the scheme A1d below:

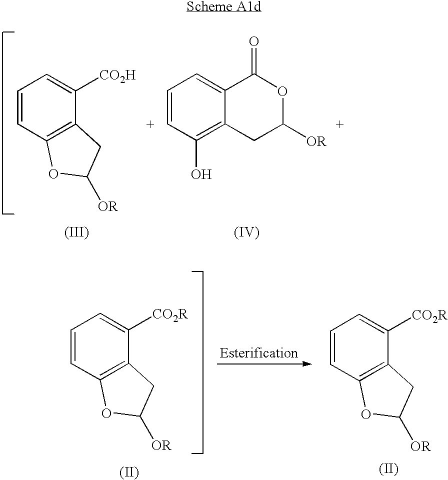

wherein R is as defined earlier.

According to another particular embodiment, this method is characterized in that the compound of formula (III) or the mixture of compounds with formulae (III), (IV) and (II) is obtained from a compound of formula (V) by an oxidation reaction, or from a mixture of a compound of formula (V) with a compound of formula (VI) and/or (VI)′ by said oxidation reaction, a reaction for acetalization and partial esterification of the compounds of formulae (VI) and/or (VI)′, performed by adding an oxidant to the reaction medium stemming from the previous step, according to the scheme A1c below:

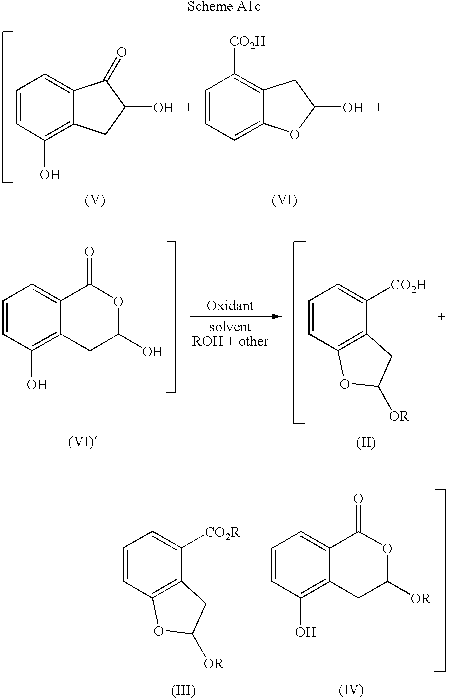

wherein R is as defined earlier.

According to a further particular embodiment, this method is characterized in that the preparation of the compound of formula (V) or of the mixture of the compound of formula (V) with a compound of formula (VI) and/or (VI)′ is obtained by the ozonolysis reaction of a compound of formula (VII), performed in a suitable solvent and at a suitable temperature for obtaining ozonides, the latter being directly reduced by adding a reducing agent for obtaining the compounds of formulae (V), (VI) and/or (VI)′ according to the scheme A1b below:

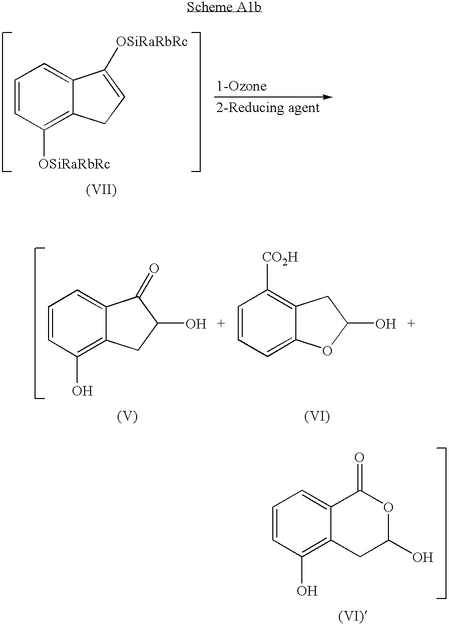

According to another particular embodiment, this method is characterized in that the compound of formula (VII) is obtained by silylation of the 4-hydroxy-indanone compound in the presence of a silylation agent, and of a catalyst, according to the scheme A1a below:

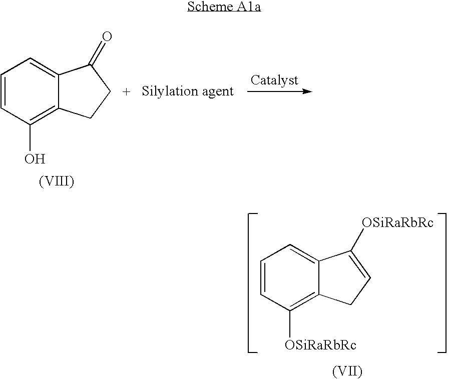

According to an independently patentable preferred embodiment, with the method of the invention, it is possible to perform a synthesis of the compound of formula (I), in 2 isolated steps, from the commercially available 4-hydroxy-indanone compound, characterized in a first isolated step by an <<in situ >> method with the four following reactions, i.e., a reaction for silylation of a ketone, an ozonolysis-reduction reaction, an oxidation reaction and an esterification reaction, and in a second isolated step, by an aromatization method.

The compound of formula (I) is then saponified, according to the scheme A below:

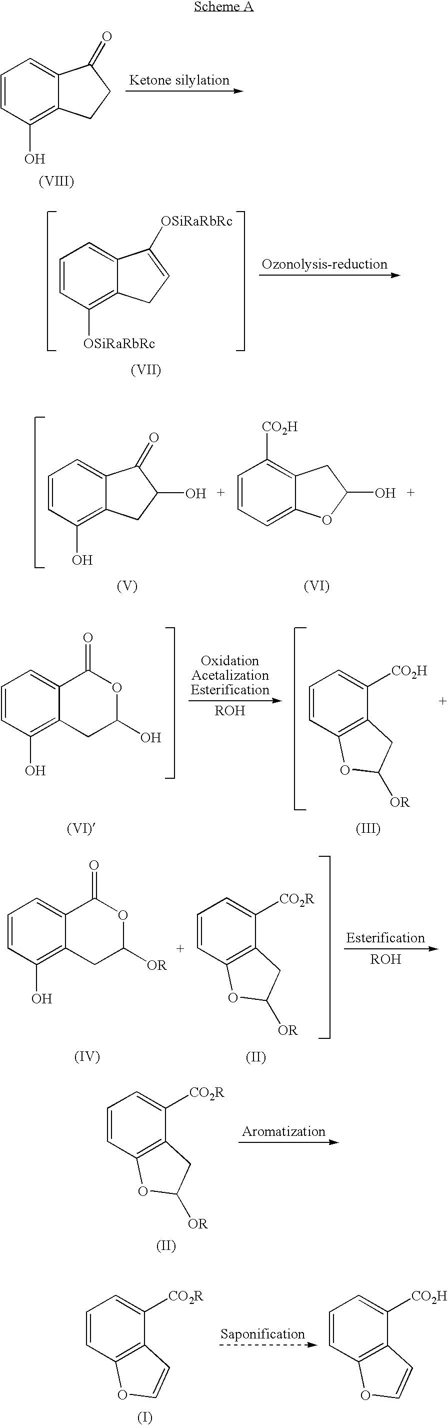

wherein:
R represents a linear or branched C₁₋₁₅ alkyl group and Ra, Rb, Rc represent a linear or branched C₁₋₁₀ alkyl group.

According to an advantageous embodiment of the invention, the invention achieves a three-step method, starting with the commercially available 4-hydroxy-indanone compound, characterized in a first step by an <<in situ>> method with ketone silylation, ozonolysis-reduction, oxidation and esterification reactions for the synthesis of the compound of formula (II), according to the scheme A1 below:

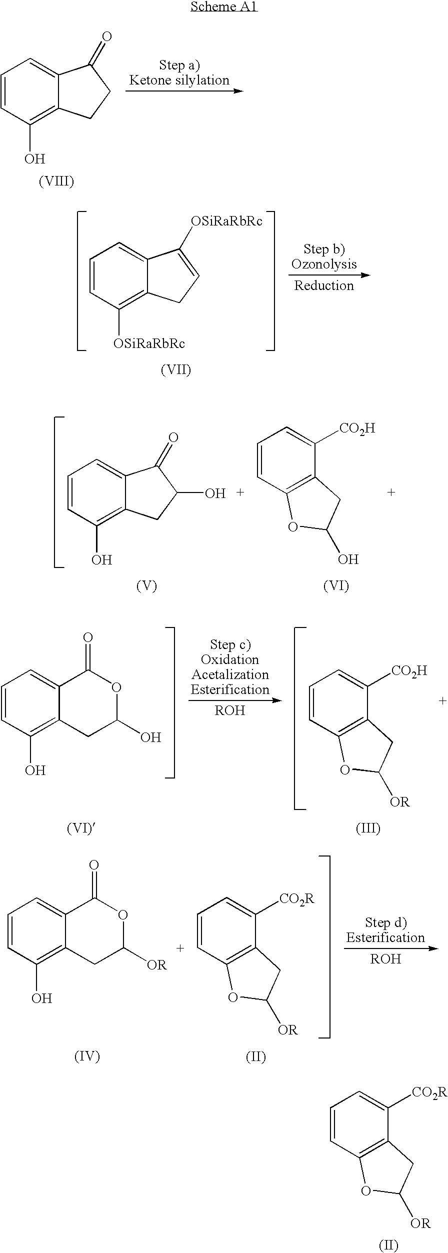

wherein R, Ra, Rb and Rc are as defined in the present description and the claims.

The method according to another advantageous embodiment of the invention applies a sequence of reactions, without isolating the formed products right up to step d, as indicated below:

a) The method according to an advantageous embodiment applies a reaction for silylation of the 4-hydroxy-indanone compound, in the presence of a silylation agent, a catalyst, according to the scheme A1a below:

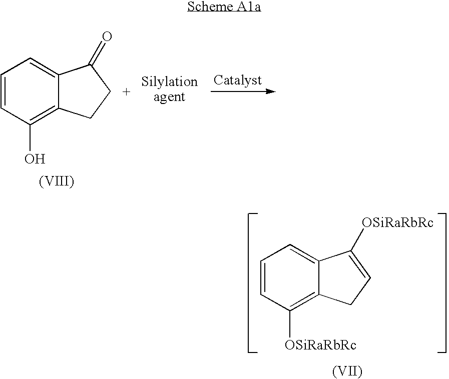

According to an alternative embodiment, the silylation agent used is represented by compounds such as N,O-bis(trimethylsilyl)acetamide (BSA); N,O-bis(trimethylsilyl)-trifluoroacetamide (BSTFA), preferentially the silylation agent is BSA.

According to an alternative embodiment, the catalyst used is represented by a quaternary ammonium halogenide (R′1R′2R′3R′4N⁺X⁻); a phosphonium halogenide, an imidazolium halogenide, preferentially the catalyst used is tetrabutylammonium bromide (TBAB).

The catalyst is used in an amount between 10 and 0.1 mol % based on the 4-hydroxy-indanone compound, preferentially the catalyst amount is about 5%.

The method may be performed in the presence or in the absence of a suitable solvent and at a temperature between 30° C. and 80° C., the method is preferentially performed in the absence of any solvent at a temperature of about 50° C.

The quantitatively obtained product of formula (VII) may either be isolated or not, the compound of formula (VII) is preferentially used as such in step b).

b) The method applies on the reaction medium stemming from a), according to an advantageous embodiment, a reaction for ozonolysis of the compound of formula (VII), in a suitable solvent and at a suitable temperature for obtaining ozonides, the latter are directly reduced by adding a reducing agent for obtaining the compounds of formulae (V), (VI) and (VI)′, according to the scheme A1b below:

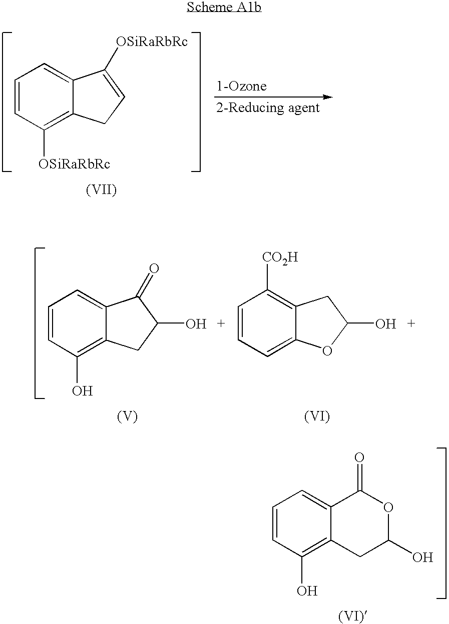

The compound of formula (VII) obtained earlier in step a), is placed in solution in a suitable solvent, the obtained solution is cooled to a temperature less than −40° C. and is then sparged with ozone. The formed intermediate ozonides are directly reduced by adding a reducing agent for forming the compound of formulae (V), (VI) and (VI)′.

The ozonolysis reaction time is directly related to the performances of the apparatuses or to the time constraints for the industrial use of the ozoner. Depending on these constraints, the reaction time will be more or less long.

According to an alternative embodiment, the reducing agent used is represented by trialkylphosphite compounds such as trimethylphosphite (MeO)₃P (TMP), dialkyl sulfides such as dimethyl sulfide Me₂S (DMS), hydrogen in the presence of a heterogenous catalyst such as Pd/C, sulfites and ethers such as dimethylsulfite (MeO)₂SO, phosphines such as triphenylphosphine ((Ph)₃P) or BINAP (2,2′-bis(diphenylphosphino)-1,1′-binaphthalene.

Preferentially, the reducing agent is TMP.

The method according to an advantageous embodiment, is performed in an organic, pure or mixed solvent, compatible with the products used in the reaction, represented by an aromatic solvent such as toluene, a halogenated solvent such as dichloromethane (CH₂Cl₂), an ester such as ethyl acetate (MeCO₂Et), an aliphatic alcohol (ROH) such as methanol (MeOH), an aromatic alcohol.

Preferentially, the solvent used in the method of scheme A1b is a mixture of toluene and methanol (MeOH) (R=Me).

The method according to scheme A1b is, according to a further advantageous embodiment, performed at a temperature between −75° C. and 0° C., preferentially the temperature is −50° C.

To arrest the exotherm of the reaction, the time for pouring in the reducing agent, is adapted more or less rapidly, depending on the cold power of the apparatuses used.

The reaction medium stemming from step 1b comprising the product of formula (VI) and the products of formulae (VI) and (VI)′, is reused as such in the following step c).

The structures of the compound of formulae (V), (VI) and (VI)′ are isolated from a sample taken from the reaction medium.

The compounds of formulae (VI) and (VI)′ are isolated by crystallization of the products while adding water to the sampled reaction medium. The precipitate is filtered and then dried.

The compound of formula (V) is isolated by performing an extraction of the preceding filtrate with an organic solvent and then by crystallization while adding water to the organic phases obtained from the extraction. The precipitate of the product is filtered and then dried.

The mixture of compounds of formula (VI) and (VI)′ was isolated as a white solid and identified by NMR analysis.

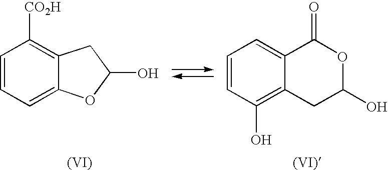

The product of formula (V) was isolated as a white solid and identified by NMR analysis. This product is novel and is claimed as such.

c) The method according to an advantageous embodiment, applies a reaction for oxidation of the compound of formula (V), for acetalization and partial esterification of the compounds of formulae (VI) and (VI)′, by adding an oxidant to the reaction medium stemming from the previous step, according to the scheme A1c below:

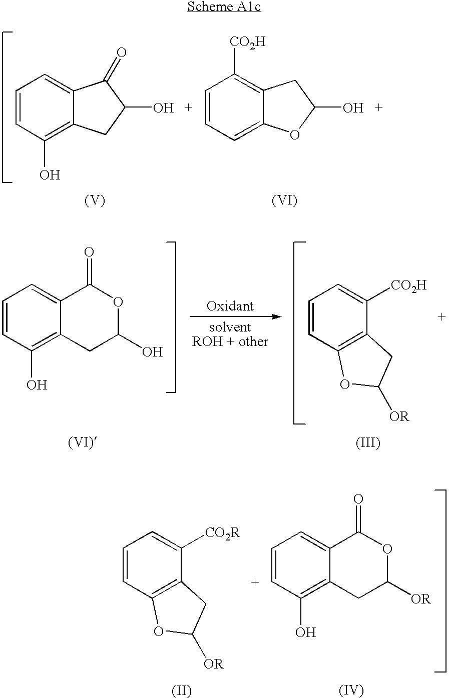

wherein R is as defined in the present description and the claims.

According to an alternative embodiment, the oxidant used is oxone or potassium peroxymonosulfate (KHSO₅), periodic acid (H₅IO₆), calcium hypochlorite (Ca(OCl)₂), nickel peroxide, hydrogen peroxide, tetrabutylammonium periodate ((n-C₄H₉)₄NIO₄), sodium bismuthate (NaBiO₃), lead tetraacetate (Pb(OAc)₄), titanium tetrachloride (Ti(Cl)₄), titanium tetraisopropoxide (Ti(OiPr)₄), sodium periodate (NaIO₄), diacetoxy iodobenzene (PhI(OAc)₂), sodium hypochlorite (NaOCl).

Preferentially, the oxidant used is oxone.

To the reaction medium obtained in step b), the oxidant is added at a temperature between −20 and 50° C., the medium is preferentially heated to a temperature of 40° C.

The reaction time is between 1 and 12 hours.

Preferentially, the solvent used in the method of scheme A1c is the one used in step A1b, more particularly a mixture of toluene and methanol (MeOH, R=Me).

The reaction medium stemming from step 1c, comprising the products of formulae (II), (III) and (IV), is reused as such in the following step d).

The compounds of formula (III) and (IV) are isolated from a sample taken from the reaction medium.

The compound of formula (III) is isolated by performing silica gel chromatography on a sample of the reaction medium, by using the eluent with the following gradient, hexane/ethyl acetate (9/1-8/2 and then 6/4).

The compound of formula (IV) is isolated by performing an extraction of the sampled reaction medium, and subsequent re-crystallization of the obtained product from isopropanol according to the standard technique of the skilled practitioner.

The products of formula (III) and (IV) were isolated as a white solid and identified by NMR analysis. These products are novel and claimed as such.

d) The method according to an advantageous embodiment of the invention, applies a reaction for esterification of the compounds of formula (III) and (IV) characterized by adding an acid to the reaction medium stemming from the preceding step, for the synthesis of the compound of formula (II), according to the scheme A1d below:

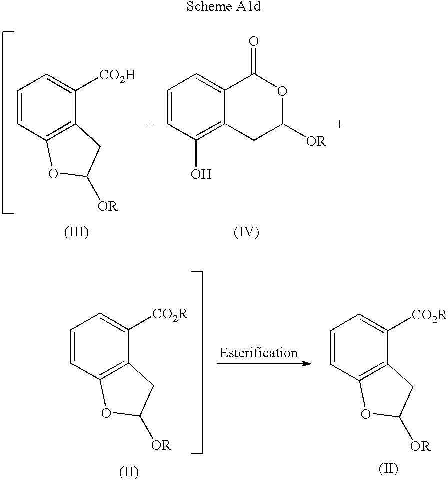

wherein R is such as defined in the present description.

To the reaction medium obtained in step c), an organic or mineral acid is added at room temperature. The medium is heated to a temperature between 20 and 50° C., the medium is preferentially heated to a temperature of about 40° C.

As an example but not as a limitation, alkyl sulfonic (AlkSO₃H) or aryl sulfonic (ArSO₃H) acids may be mentioned among the organic acids; halogenated acids (HI, HCl, HBr), sulfuric acid (H₂SO₄), perchloric acid (HClO₄) may be mentioned among the mineral acids.

Preferentially, the acid used is methane sulfonic acid (MeSO₃H).

The compound of formula (II) is isolated from a sample taken from the reaction medium.

The compound of formula (II) is isolated by performing an extraction of the sampled reaction medium and then by dry concentration of the organic phase according to standard techniques known to the skilled practitioner.

The product of formula (II) is isolated as a white solid. This product is novel and claimed as such.

The yield for obtaining the compound of formula (II) from 4-hydroxy-indanone according to steps a), b), c) and d) by using the preferentially mentioned reagents, solvents and temperatures, is 65%.

According to an advantageous embodiment of the invention, the invention achieves a two-step method for forming the compound of formula (I) from the commercially available 4-hydroxy-indanone compound, characterized in a second step, by a reaction for aromatizing the compound of formula (II) in order to synthesize the compound of formula (I), according to the scheme A2 below:

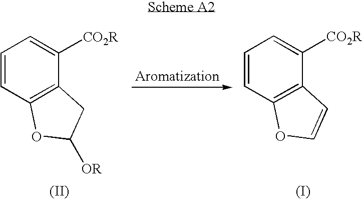

wherein R is such as defined in the present description and the claims.

The aromatization reaction is performed by adding an organic or mineral acid.

As an example but not as a limitation, alkyl sulfonic (AlkSO₃H) or aryl sulfonic (ArSO₃H) acids may be mentioned among the organic acids; halogenated acids (HI, HCl, HBr), sulphuric acid (H₂SO₄), phosphoric acid (H₃PO₄) may be mentioned among the mineral acids.

Preferentially, the acid used is methane sulfonic acid (MeSO₃H).

The reaction is performed at room temperature, preferentially at 20° C.

The compound of formula (I) is isolated according to standard techniques known to the skilled practitioner.

The product of formula (I) is isolated either as a white solid or as an organic solution.

Preferentially, the compound of formula (I) is isolated as a toluene solution which is directly used in a saponification reaction according to the scheme B below:

The saponification reaction is performed according to the method described in the article J. Med. Chem., 1995, p 3094-3105.

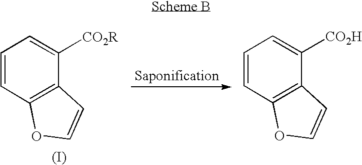

The invention according to a second aspect covers the following novel products:

A) The Compound of Formula (V).

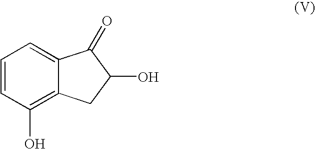

B) The Compound of Formula (IV).

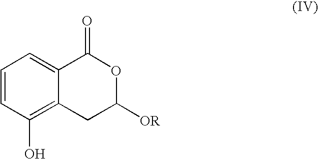

wherein R represents a linear or branched C₁₋₁₅ alkyl group.

C) The Compound of Formula (III).

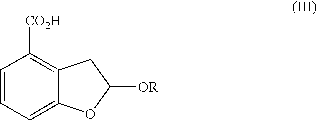

wherein R represents a linear or branched C₁₋₁₅ alkyl group.

D) The Compound of Formula (II).

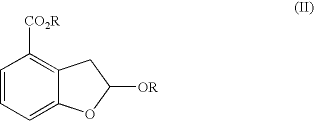

wherein R represents a linear or branched C₁₋₁₅ alkyl group.

According to a third aspect, the invention further covers the use of the compound of formula (I) as obtained by the method defined earlier or as a result of the following description with reference to the exemplary embodiments which are an integral part of the invention, in order to achieve synthesis of 4-benzofuran-carboxylic acid.

According to a fourth aspect, the invention further covers the use of at least one compound of formula (II), (III), (IV) or (V) as defined above or in the following description, in order to achieve synthesis of 4-benzofuran-carboxylic acid.

It should be noted that any technical feature which appears to be novel with respect to any STATE OF THE ART is claimed as such, in its technical function and as a general technical means including all equivalent technical means, as this may be well understood by the skilled practitioner.

In the examples, percentages are given by weight/weight, temperature is given in degrees Celsius, pressure is atmospheric pressure, unless indicated otherwise.

### DESCRIPTION OF THE EXAMPLES OF THE INVENTION

To facilitate comprehension, the nomenclature of the products, reagents and solvents, is the international nomenclature or the one currently used by the skilled practitioner.

Scheme A1: Examples 1 to 4.

### Example 1

### Silylation

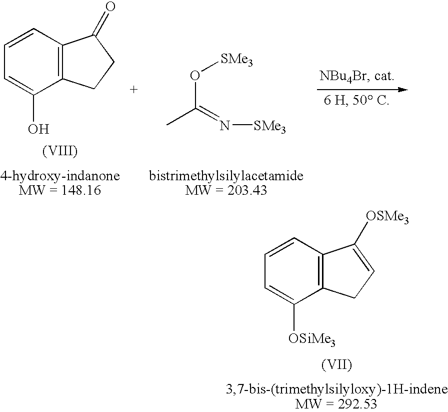

Unless indicated otherwise, all the volumes and equivalences are calculated from 4-hydroxyindanone.

Under nitrogen, to 3.3 kg of bis-trimethylsilyl acetamide (98.09% w/w; 2.38 mol/mol), 110 g of tetrabutylammonium bromide (5 mol %) are added; followed by fractionated addition of 1 kg of 4-hydroxy-indanone. The medium is heated for 6 hours at 50° C., the formed dimethylsilyl acetamide partially sublimates during the reaction. The reaction medium is cooled to room temperature and is diluted in 2 volumes of toluene. The solution is then introduced as such to the following step. TLC analysis indicates complete conversion.

### TLC, Hex/EA 8/2 Rf=0.96 (UV)

¹H NMR (200 MHz, CDCl₃) δ (ppm): 0.1-0.5 (OSiCH₃); 3.05 (2H, m, CH₂); 5.3 (1H, m, CH); 6.5 (1H, d, ³J=7.8 Hz, CH); 6.9 (1H, d, ³J=7.4 Hz, CH); 7.05 (1H, t, ³J=7.4 Hz, CH).

¹³C NMR (50 MHz, CDCl₃) δ (ppm): 0.1-0.6 (OSiCH₃); 105.7; 111.9; 116.5; 127.3; 132.1; 143.7; 150.4; 153.1; 175.9.

### GPC/MS (IE): [M]⁺=292

### Example 2

### Ozonolysis

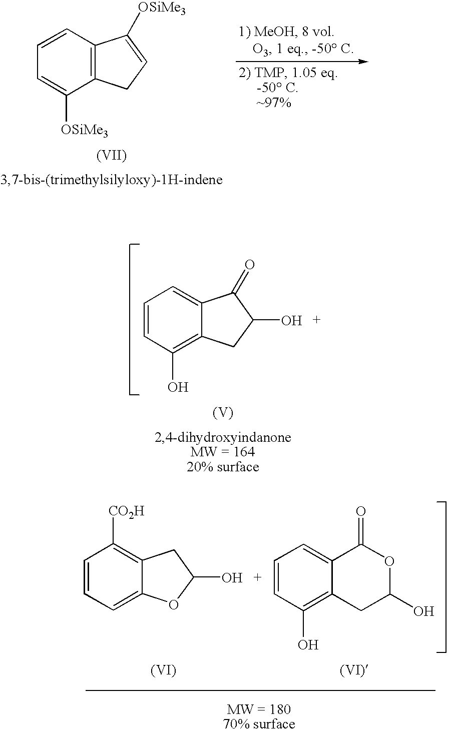

Unless indicated otherwise, all the volumes and equivalences are calculated from 4-hydroxyindanone.

The toluene solution of 3,7-bis-trimethylsilyloxy-1-H-indene is added to 8 L of methanol cooled to −50° C. Ozonized air bubbling is applied to this solution until HPLC analysis indicates complete conversion. The ozonides are then reduced at −50° C. with 879 g of trimethylphosphite (1.05 mol/mol). The solution is then introduced as such to the following step.

Two majority products are identified by HPLC: acid forms comprising at least two products and dihydroxyindanone in a respective surface proportion of 70/20.

From the crude medium, water is added. The formed precipitate is filtered and dried. A white solid is obtained, corresponding to the structures below inferred from ¹H NMR and ¹³C NMR analysis:

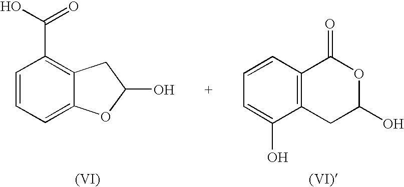

¹H NMR (200 MHz, DMSO-d6) δ (ppm): 2.8-3.6 (2H, m, CH₂), 5.8 (1H, s, CH), 6.0 (1H, s, CH), 6.9 (5H, m, CH), 7.7 (1H, s, OH), 10.1 (1H, s, OH), 12.9 (1H, s, COOH).

¹³C NMR (50 MHz, DMSO-d6) δ (ppm): 28.2 (CH₂); 38.7 (CH₂); 95.0; 100.3; 114.7; 115-117; 121.8; 128-129; 157.1; 177.4.

The previous filtrate is extracted. Water is added to the organic phases, the formed precipitate is filtered and dried. A white solid is obtained, corresponding to the compound of formula (V), 2,4-dihydroxyindanone.

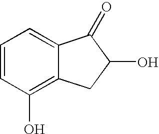

¹H NMR (200 MHz, DMSO-d6) δ(ppm): 3.4-3.7 (2H, 2m, CH₂), 4.17 (1H, s, CH), 5.82 (1H, s, OH), 7.43-7.66 (3H, m, CH), 9.90 (1H, s, OH).

¹³C NMR (50 MHz, DMSO-d6) δ(ppm): 32.5 (CH₂); 73.7 (CH); 114.3; 120.2 and 129.5 (CAr); 135.9; 136.7 and 154.9 (quaternary CAr); 206.5 (CO).

### Mp (° C.): 170.84° C.

### Example 3

### Oxidation

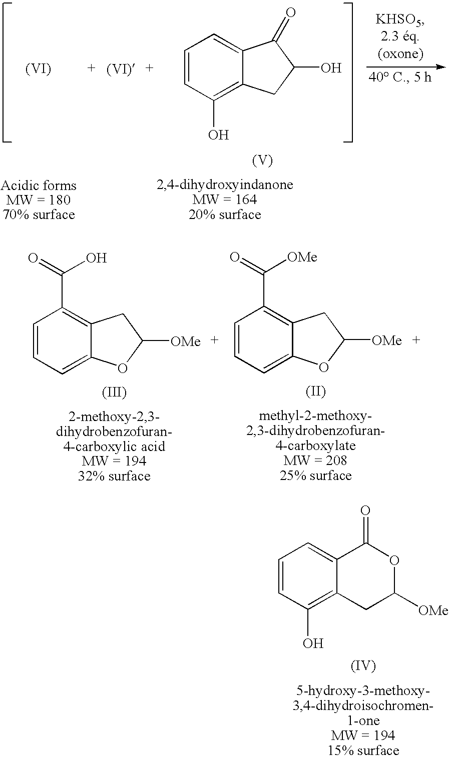

Unless mentioned otherwise, all the volumes are calculated from 4-hydroxyindanone.

The equivalents are calculated from 4-hydroxyindanone and from TMP.

At 20° C., feed 1.7 kg of oxone (2.3 mol in KHSO₅) onto the previous solution while letting the temperature vary. The medium is then stirred at 40° C. for 5 hours. The salts are filtered at room temperature and washed 4 times with 2 volumes of methanol (0.6 L). The obtained majority product is 2-methoxy-2,3-dihydrobenzofuran-4-carboxylic acid mixed with 5-hydroxy-3-methoxy-3,4-dihydroisochromen-1-one and 2-methoxy-2,3-dihydrobenzofuran-4-carboxylic acid methyl ester.

The solution is then introduced as such to the following step.

Silica gel column chromatography is performed on a sample from the solution (eluent with a gradient: hexane/ethyl acetate (9/1-8/2 and then 6/4)).

A white solid is obtained, corresponding to the compound of formula (III), 2-methoxy-2,3-dihydrobenzofuran-4-carboxylic acid:

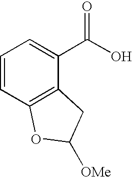

¹H NMR (200 MHz, DMSO-d6) δ (ppm): 3.41-3.25 et 3.50-3.59 (2H, m, CH₂), 5.70-5.73 (1H, m, CH), 7.05-7.45 (3H, m, CH), 12.98 (1H, s, OH).

¹³C NMR (50 MHz, DMSO-d6) δ (ppm): 37.4 (CH₃); 55.2 (CH₂); 106.9 (CH); (113.3; 127.5; 127.7 et 127.9) (C Ar); 158.2; 166.9 (CO).

### Mp (° C.): 181.73° C.

The reaction medium is extracted and then re-crystallization is performed in isopropanol. A white solid is obtained, corresponding to the compound of formula (IV), 5-hydroxy-3-methoxy-3,4-dihydroisochromen-1-one.

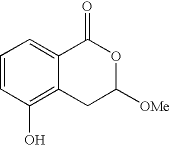

¹H NMR (200 MHz, CD₃OD) δ(ppm): 3.54-3.56 (2H, m, CH₂), 3.96 (3H, s, CH₃), 5.86 (1H, t, ³J=3.89 Hz, CH), 7.45 (1H, d, ³J=8.14 Hz, CH), 7.59 (1H, t, ³J=7.79 Hz, CH), 7.94 (1H, d, ³J=7.08 Hz, CH).

¹³C NMR (50 MHz, CD₃OD) δ(ppm): 26.7 (CH₂); 56.1 (CH₃); 102.2 (CH); (119.8; 120.2; 122.9; 124.8; 127.3) (CAr); 153.8 (CO).

GPC/MS (IE): [M+H]⁺=195; ion fragments: 177; 163; 134; 106; 91; 77; 63; 51; 39; 28.

### Example 4

### Esterification

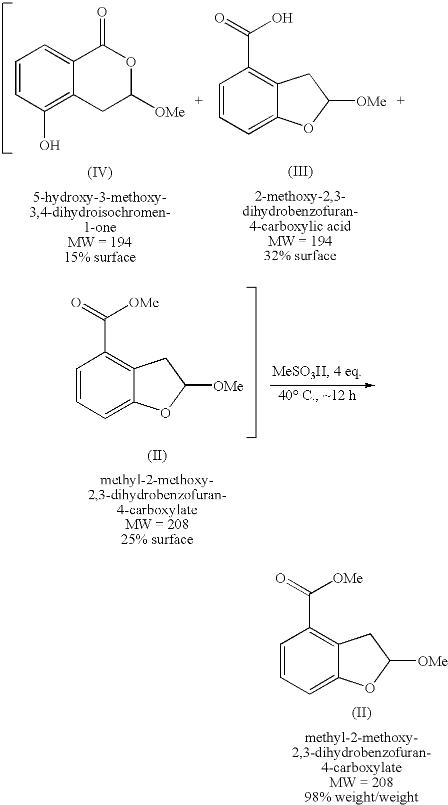

Unless mentioned otherwise, all the volumes and equivalences are calculated from 4-hydroxyindanone.

At room temperature, 2.58 kg of methane sulfonic acid (4 mol/mol) are added to the previous solution so as not to exceed 40° C. The medium is then heated to 40° C. for about 12 hours. The reaction medium is then cooled to 30° C. and methoxytrimethylsilane is distilled in a slight vacuum. The vacuum is then increased to as to totally distil the methanol right up to a residual volume of about 5 L. 3 L of methanol (3 volumes) are added to the reaction medium at 30° C., followed by 3 L of toluene (3 volumes). The medium is then cooled to 20° C. and 4 L of water (4 volumes) are then added while maintaining the temperature of the medium at 20° C. The phases are separated and the aqueous phase is extracted twice with 3 L of toluene (2×3 volumes). The collected organic phases are washed with 4×2 L of water (4×2 volumes) (return to a pH=˜5), and then with 2×2 L of 1 N soda (2×2 volumes), finally with 3×2 L of water (2 volumes) until the pH returns to pH=˜8. The insolubles are kept with the organic phase which is clarified on Clarcel®.

The water/toluene azeotrope is distilled in vacuo (T=85° C., 760 mm Hg), and the toluene is then distilled in vacua at 45±5° C. up to a residual volume of about 3 L. 910 g of 2-methoxy-2,3-dihydrobenzofuran-4-carboxylic acid methyl ester are obtained in solution in the toluene with a 65% yield from 4-hydroxyindanone.

Extraction and concentration are performed on a sample of the solution.

A white solid is obtained corresponding to the compound of formula (II), 2-methoxy-2,3-dihydrobenzofuran-4-carboxylic acid methyl ester.

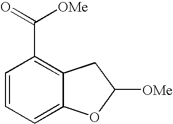

¹H NMR (200 MHz, DMSO-d6) δ (ppm): 3.84 (3H, s, CH₃); 4.24 (3H, s, CH₃); 6.15-6.18 (1H, m, CH); 7.52 (1H, d, ³J=7.79 Hz, CH), 7.69 (1H, t, ³J=7.79 Hz, CH), 7.86 (1H, d, ³J=7.79 Hz, CH).

GPC/MS (EI): [M]⁺=208; ion fragments: 177; 165; 145; 134; 105; 89; 77; 63;40.

Scheme A2.

### Example 5

### Aromatization

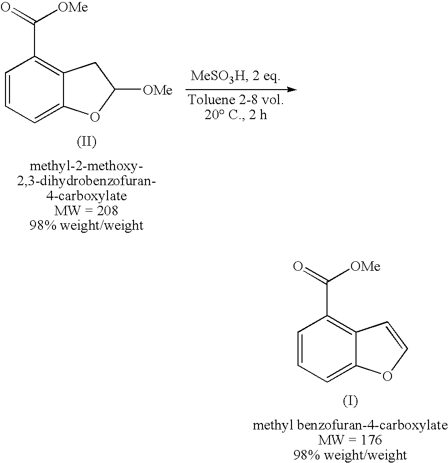

Unless mentioned otherwise, all the volumes are calculated from 2-methoxy-2,3-dihydrobenzofuran-4-carboxylic acid methyl ester (II).

841 g of methane sulfonic acid (8.75 mol, 2 mol/mol) are added to the previous toluene solution while maintaining the temperature at 20° C.

The reaction medium is stirred at 20° C. for 2 hours. The reaction medium is then cooled to 10° C., 3 water volumes are added while maintaining the temperature of the medium at 10° C.

The phases are separated and the lower aqueous phase is then extracted with 2 volumes of toluene. The collected toluene phases are washed with 2×2 volumes of water and then with 2×2 volumes of 1N soda.

The insolubles formed in the basic medium are left in the organic phase, the toluene phase is clarified and introduced as such to the following step. 747 g of 4-benzofuran-carboxylic acid methyl ester are obtained in solution in toluene with a 98% yield.

The reaction medium is extracted and then concentrated.

A white solid is obtained corresponding to the compound of formula (I), 4-benzofuran-carboxylic acid methyl ester.

¹H NMR (200 MHz, CDCl₃) δ (ppm): 3.97 (3H, s, CH₃); 7.33-7.36 (1H, m, CH), 7.66-7.72 (1H, m, CH), 7.95-7.98 (1H, m, CH).

¹³C NMR (50 MHz, CDCl₃) δ (ppm): 51.9; 107.7; 115.8; 122.7; 123.5; 125.3; 127.8; 146.4; 155.1; 166.7.

GPC/MS (EI): [M+H]⁺=177; ion fragments: 145; 133; 105; 91; 59.
Mp (° C.): 40.1° C.

What is claimed is:

1. A method for the synthesis of 4-benzofuran-carboxylic acid or alkyl ester thereof of formula (I), comprising performing a reaction for aromatization of a compound of formula (II) to synthetize said compound of formula (I), according to the scheme A2 below: 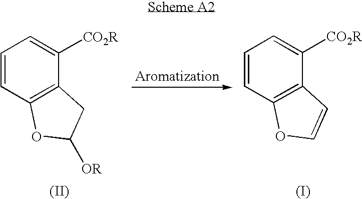
wherein R independently represents hydrogen, a linear C₁₋₁₅ alkyl group, and a branched C₁₋₁₅ alkyl group.

2. The method of claim 1, comprising preparing the compound of formula (II) by esterifying a compound of formula (III) or a mixture of compounds of formulae (III), (IV) and (II), stemming from a prior reaction, by adding an acid to the reaction medium, according to the scheme A1d below: 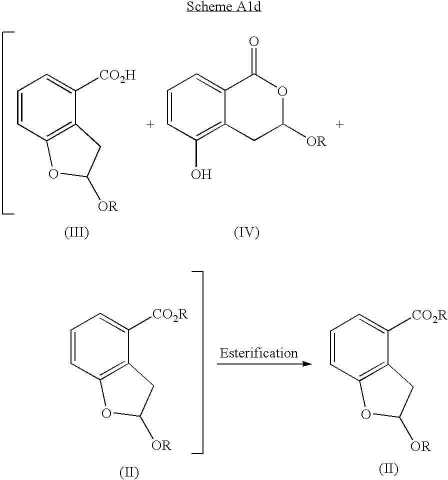
wherein R is such as defined in claim 1.

3. The method of claim 2, comprising preparing the compound of formula (III) or the mixture of compounds of formulae (III), (IV) and (II) from:
a) either a compound of formula (V) by an oxidation reaction,
b) or from a mixture of a compound of formula (V) with a compound of formula (VI) and/or (VI)′by said oxidation reaction,
by acetylization and partial esterification reaction of the compounds of formulae (VI) and (VI)′,
performed by adding an oxidant to the reaction medium stemming from the previous step, according to the scheme A1c below: 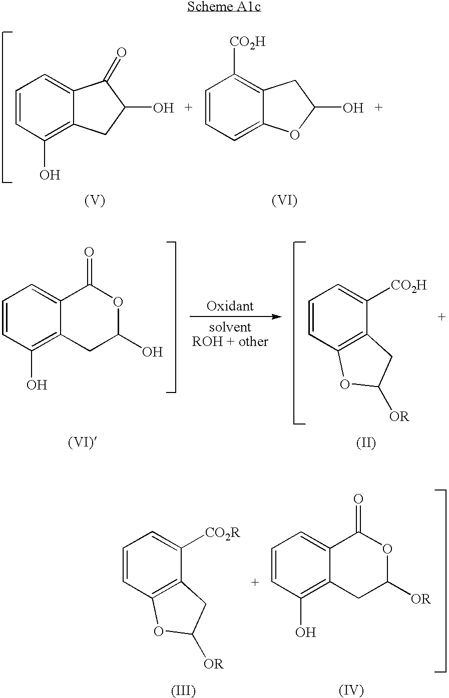
wherein R is as defined in claim 1.

4. The method of claim 3, comprising obtaining the compound of formula (V) or of the mixture of the compound of formula (V) with the compound of formula (VI) and the compound of formula(VI)′ by an ozonolysis reaction of a compound of formula (VII), performed in a suitable solvent at a suitable temperature for obtaining ozonides, and directly reducing said ozonides by adding a reducing agent to obtain said compounds of formulae (V), (VI) and/or (VI)′, according to the scheme A1b below:

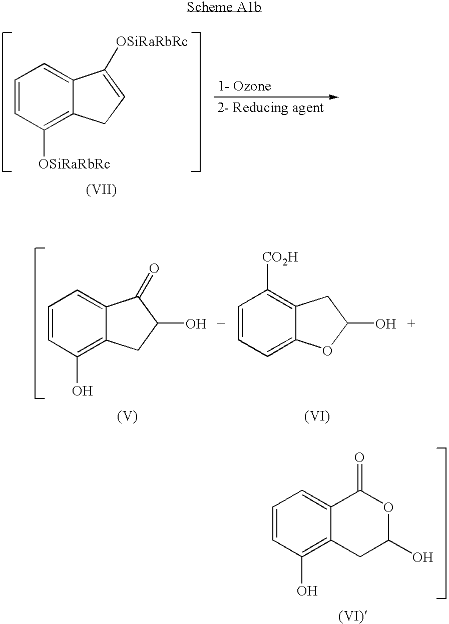

5. The method of claim 4, comprising preparing the compound of formula (VII) by silylation of a 4-hydroxy-indanone compound of formula (VIII) in the presence of a silylation agent, and of a catalyst, according to the scheme A1a below:

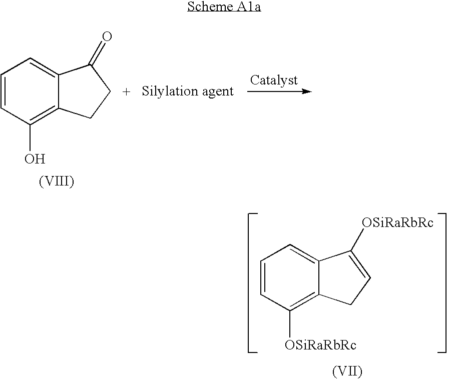

6. A method for preparing 4-benzofuran-carboxylic acid or alkyl ester thereof of formula (I), in two steps, from a 4-hydroxy-indanone compound, comprising:
i) a first step with an <<in situ >>method with four successive reactions comprising:
a) a ketone silylation,
b) an ozonolysis-reduction,
c) an oxidation and
d) an esterification; and
ii) a second step with an aromatization method, according to the scheme below: 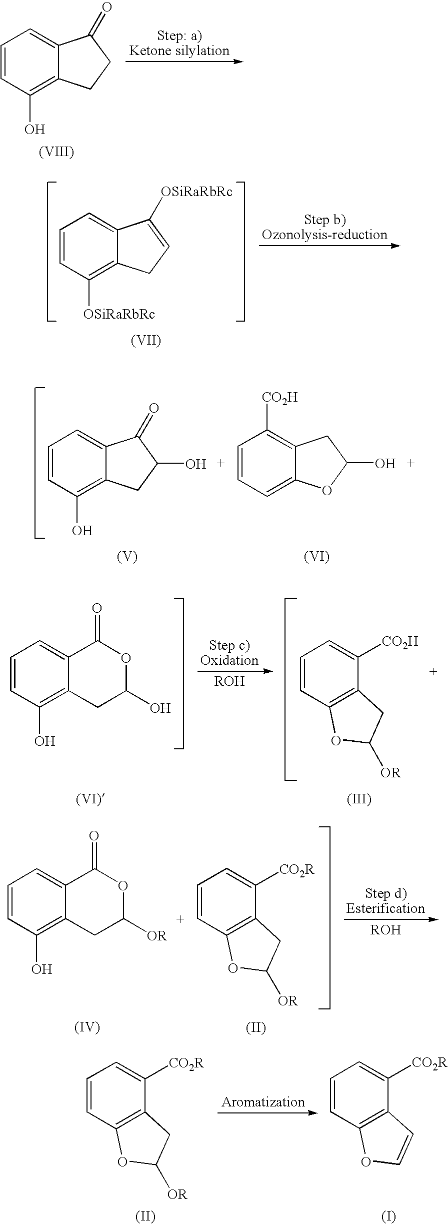
wherein R represents a linear C₁₋₁₅ alkyl group, a branched C₁₋₁₅ alkyl group; and Ra, Rb, Rc represent a linear C₁₋₁₀ alkyl group, a branched C₁₋₁₀ alkyl group.

7. The method of claim 6, characterized in that a), b), c) and d) are applied without isolating the formed products right up to step d.

8. The method of claim 5, wherein the silylation agent is N,O-bis(trimethylsilyl)acetamide (BSA).

9. The method of claim 5, wherein the catalyst is a quaternary ammonium halogenide.

10. The method of claim 5, wherein the catalyst is tetrabutylammonium bromide (TBAB).

11. The method of claim 5, wherein the reducing agent is trimethylphosphite (MeO)₃P (TMP).

12. The method of claim 3, wherein the oxidant is potassium peroxymonosulfate (KHSO₅, oxone).

13. The method of claim 2, wherein the acid is an organic acid.

14. The method of claim 2, wherein the acid is a mineral acid.

15. The method of claim 2, wherein the acid is methane sulfonic acid (MeSO₃H).

16. The method of claim 1, wherein the aromatization reaction is performed by adding:
a) either an organic acid selected from the group consisting of an alkyl sulfonic acid (AlkSO₃H); and an aryl sulfonic acid (ArSO₃H),
b) a mineral acid selected from the group consisting of a halogenated acid sulphuric acid (H₂SO₄) and phosphoric acid (H₃PO₄).

17. The method of claim 16, wherein said halogenated acid is selected from HI, HCl, and HBr.

18. A compound of formula (V):

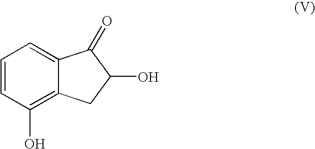

19. A compound of formula (IV): 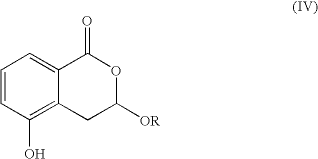
wherein R represents a linear C₁₋₁₅ alkyl group, a branched C₁₋₁₅ alkyl group.

20. A compound of formula (III). 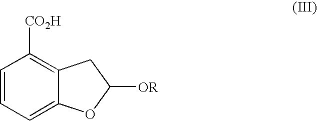
wherein R represents a linear C₁₋₁₅ alkyl group, a branched C₁₋₁₅ alkyl group.

21. A compound of formula (II). 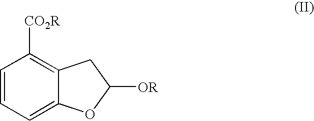
wherein R represents a linear C₁₋₁₅ alkyl group, a branched C₁₋₁₅ alkyl group.

22. A method of synthesis of 4-benzofuran-carboxylic acid comprising preparing said 4-benzofuran-carboxylic acid from a compound of formula (II), as defined in claim 21.

23. A method of synthesis of 4-benzofuran-carboxylic acid comprising preparing said 4-benzofuran-carboxylic acid from a compound of formula (III), as defined in claim 20.

24. A method of synthesis of 4-benzofuran-carboxylic acid comprising preparing said 4-benzofuran-carboxylic acid from a compound of formula (IV) as defined in claim 19.

25. A method of synthesis of 4-benzofuran-carboxylic acid comprising preparing said 4-benzofuran-carboxylic acid from a compound of formula (V) as defined in claim 18.
